(19) United States
(12) Patent Application Publication
(10) Pub. No.: US 2009/0188508 A1
(43) Pub. Date: Jul. 30, 2009

(54) Disposable medical mouthpiece appliance

(76) Inventor: A. James Smith, JR., Naples, FL (US)

Correspondence Address: A. James Smith Jr., 4021 Gulf Shore Blvd. #1905, Naples, FL, 34103, US

(21) Appl. No.: 12/011,622

(22) Filed: Jan. 29, 2008

Related U.S. Application Data
(63) Continuation-in-part of application No. 11/900,887, now abandoned.

(51) Int. Cl. A61M 16/00

(52) U.S. Cl. 128/207.14

(57) ABSTRACT

A medical appliance in the form of a pliable mouth piece in the shape of a funnel having a cylindrical section attached thereto. The pliable mouth piece is made of a robo rubber. An additional rigid tube may be inserted into the cylindrical section. The pliable appliance may be used with patients having a stoma to apply air by blowing into the mouth piece. The mouth piece may also be a part of a spirometer to test the lung capacity of people. The medical appliance may be used in patients who had a tracheotomy and by EMS technicians who perform a tracheotomy in an emergency.

This application is a Continuation-In-Part of application Ser. No. 11/900,887 filed on Sep. 14, 2007

### BACKGROUND OF THE INVENTION

Devices are known that are used on patients that have lung problems such as the use of spirometers that measure a patients lung capacity. There is a simple hard mouthpiece into which a patient will blow. For patients who breath through a stoma, all that is being done is to put the tube over the stoma and it has been found that a hard tube would either induce leakage of air during the process of blowing air into the tube or would bruise and otherwise damage the skin if pressed hard enough to prevent such a leakage.

There are also patients who for physical or psychological reasons cannot make a tight seal with their lips making accurate spirometer readings problematic.

### BRIEF DESCRIPTION OF THE INVENTION

The inventive concept consists of pliable material in the form of a cylindrical tube section that has a funnel attached at one end thereof. It is a pliable material that easily conforms to various surfaces such as a neck, a throat or a mouth. The cylindrical tube section has an inner lip and is formed in such a manner so that it can receive a rigid tube of the spirometer. The pliable funnel is placed over the stoma which transports the exhalations to the spirometer. The pliable funnel is placed over a stoma or around the lips. The material used in the demo model of this concept is Reporubber® which can be an irritant to the skin and is used in this embodiment only for illustration.

### BRIEF DESCRIPTION OF THE DRAWINGS

FIG. 1 is a perspective of the medical appliance including a funnel and a removable tubular insert;

FIG. 2 is a cross section through appliance of FIG. 1;

FIG. 3 is an end view of the appliance of FIG. 1 into the funnel end;

### DETAILED DESCRIPTION OF THE INVENTION

The primary use of this invention is the use as a disposable mouthpiece which attaches at the cylinder end to the “intake port” of the spirometer. The funnel end is placed around the stoma to form a seal.

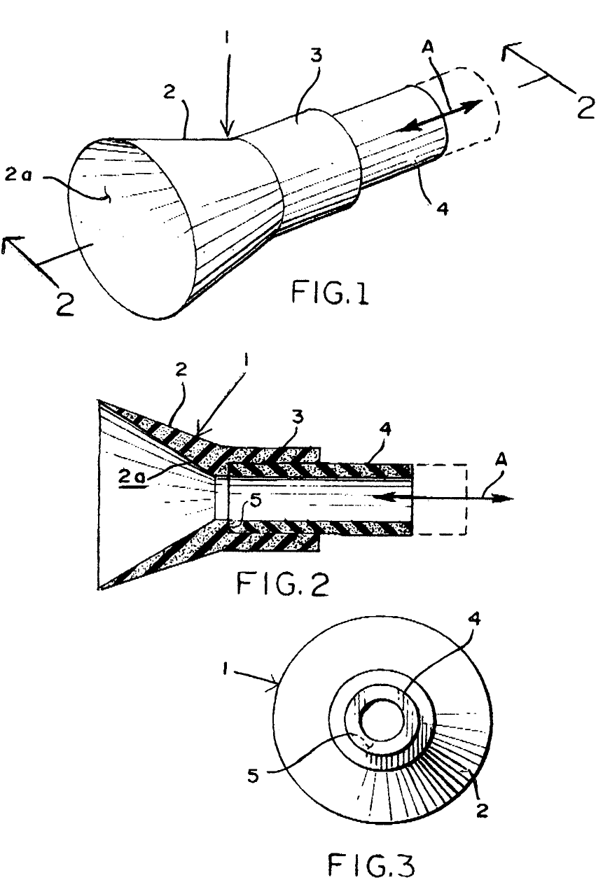

FIG. 1 is a perspective view of the medical appliance 1 in the form of a mouth piece having a funnel 2 at one end thereof and a cylindrical section 3 at another end. The cylindrical end 3 can be inserted into a stoma and the funnel end 2 can be placed over the mouth so that a person can blow into the funnel without any leaks. The procedure can be reversed by placing the funnel over the stoma and by blowing into the cylindrical section 3. To remove any discomfort to the person treating the patient, a removable tube 4 can be inserted into the cylindrical section of the appliance 1. The removability of the tube 4 is indicated by the arrow A.

As can be seen from FIG. 2, there is an offset 5 within the funnel 2 that will form a seat for the removable insert tube. The removable tube 4 can be made of a rigid plastic. Also in FIG. 2 it can be seen that that the interior of the funnel itself is tapered as at 2a. This feature contributes to the pliability of the funnel. It aids adapting itself to the various contours of surfaces to which the funnel is applied.

FIG. 3 Is an end view of the funnel 2 by looking into it. The same reference characters that were applied to FIGS. 1 and 2 are used in this view.

### Operation

It can be seen that the inventive medical appliance has many different applications. For example, for patients who had a tracheotomy, the funnel shaped piece can by placed over the incision in the form of a hole to achieve an air tight fit. Another recommended use is for the EMS. If there is a blockage in a person's throat, the attending medical technician must perform an incision. The problem is not the incision but the rigid tube that is being inserted. The rigid tube could damage the surrounding tissue. By placing the funnel shaped section 2 over the incision, an airtight seal is obtained achieving the desired results.

It is foreseen that the appliance could be used and might be quite helpful during the process of a mouth-to-mouth resuscitation. In this situation it would be useful to attach a mouth piece 2 to each end of the rigid 4 to thereby avoid a direct contact of the mouths of both the patient as well as the medical technician which would greatly enhance the hygiene between the two persons.

It is pointed out that the appliance can be manufactured at a low cost and is easily disposable. The medical appliance would be delivered to its final destination or locality of use in a sterile package and will remain sterile until it is used or the package is opened.

What I claim is:

1. A medical appliance including a pliable mouth piece in the form of a funnel and a cylindrical tube attached to said funnel.

2. The medical appliance of claim 1, wherein the thickness of said funnel is tapered inwardly.

3. The medical appliance of claim 1, including a removable rigid tube to be inserted into said cylindrical tube.

4. The medical appliance of claim 3, wherein said funnel has an interior offset as a seat for said rigid tube.

5. The medical appliance of claim 1, wherein the material of said pliable mouth piece is a robo-rubber.
